(19) United States
(12) Patent Application Publication
(10) Pub. No.: US 2011/0077484 A1
(43) Pub. Date: Mar. 31, 2011

(54) Systems And Methods For Identifying Non-Corrupted Signal Segments For Use In Determining Physiological Parameters

(75) Inventors: Braddon M. Van Slyke, Arvada, CO (US); Paul Stanley Addison, Edinburgh (GB); Scott McGonigle, Edinburgh, Midolothian (GB); James Nicholas Watson, Dunfermline (GB)

(73) Assignee: Nellcor Puritan Bennett Ireland, Mervue (IE)

(21) Appl. No.: 12/570,388

(22) Filed: Sep. 30, 2009

(51) Int. Cl. A61B 5/1455; G06N 3/02; G06N 3/08

(52) U.S. Cl. 600/324; 706/20; 706/25

(57) ABSTRACT

According to embodiments, non-corrupted signal segments are detected by a data modeling processor implementing an artificial neural network. The neural network may be trained to detect artifact in the signal (e.g., a PPG signal or some wavelet representation of a PPG signal) and gate valid signal segments for use in determining physiological parameters, such as, for example, pulse rate, oxygen saturation, pulse rate, respiration rate, and respiratory effort. When an artifact is detected, previously received known-good signal segments may be buffered and replace the signal segment or segments containing artifact. A regression analysis may also be performed in order to extrapolate new data from previously received known-good signal segments. In this way, more accurate and reliable physiological parameters may be determined.

### SUMMARY

The present disclosure relates to signal processing and, more particularly, the present disclosure relates to processing, for example, a photoplethysmograph (PPG) signal to determine physiological parameters of a patient.

As described in more detail below, a pulse oximeter may be used to determine oxygen saturation, pulse rate, and other physiological parameters by an analysis of an optically sensed plethysmograph. The oximeter may pass light using a light source through blood perfused tissue and photoelectrically sense the absorption of light in the tissue.

The optical signal through the tissue, however, can be degraded by many sources of noise. One source of noise may include ambient light which reaches the light detector. Another source of noise may include electromagnetic coupling or interference from other electronic instruments. Movement of the patient also introduces noise and may affect the optical signal. For example, the contact between the light detector and the skin (or the light emitter and the skin) can be temporarily disrupted when a patient's movement causes either the detector or emitter to move temporarily away from the skin. In addition, since blood is a fluid, it responds differently than the surrounding tissue to inertial effects, thus resulting in momentary changes in volume at the point to which the oximeter probe is attached. This may introduce yet another source of noise in the optical signal, resulting in degradation of the optical signal. Any of the aforementioned sources of noise (as well as other types of noise) may result in the presence of artifact in the detected optical signal.

As described in U.S. patent application Ser. No. 12/245,336, which is hereby incorporated by reference herein in its entirety, some artifacts appearing in a scalogram derived from a continuous wavelet transform of a PPG signal may be masked and filtered from the scalogram, leaving only the portions of the scalogram that are free from artifact. One or more physiological parameters may then be determined from the scalogram with the artifact regions removed. In this way, more accurate physiological parameters may be determined.

In an embodiment, regions free from artifact may be identified in a scalogram and flagged (or “gated”) for use in determining physiological parameters, such as oxygen saturation, pulse rate, respiration rate, respiratory effort, and blood pressure. The artifact-free regions may be identified or gated in real-time as the underlying signal is collected (e.g., from a pulse oximetry system). Real-time identification of non-corrupted or artifact-free scalogram segments may allow for continuous output of a patient's physiological parameters derived, at least in part, from the non-corrupted or artifact-free segments. Previously known values of the patient's physiological parameters may be buffered until a suitable artifact-free region is detected for an updated valid measurement.

In an embodiment, a data modeling processor includes a non-linear statistical data modeling module that identifies valid scalogram segments. The modeling processor (which may take the form of an artificial neural network (ANN) in some embodiments) may be trained to identify scalogram segments that are valid for use in determining physiological parameters. For example, in some embodiments, the data modeling processor may perform one or more regression analyses (e.g., using linear or nonlinear regression techniques) on the input data. Valid signal segments may then be identified and may include segments not identified as having artifact (or having less than some threshold level of artifact), segments that are not stale (e.g., segments collected within some user-defined freshness threshold), or segments that are both free from artifact and not stale. The valid signal segments may then be used to determine one or more physiological parameters while the invalid signal segments may be discarded or removed from the scalogram (e.g., the invalid signal segments may be weighted to zero). One or more previously valid physiological parameter measurements may be held or buffered until a new valid measurement is determined from a useable portion of valid signal segments.

In an embodiment, the data modeling processor may operate directly on the detected signal itself (e.g., a PPG signal) or some transform of the detected signal (e.g., a continuous wavelet transform of a PPG signal). In some embodiments, the data modeling processor may also operate on a scalogram derived from the transformed signal, a wavelet ratio surface, the real part of the wavelet transform, the imaginary part of the wavelet transform, the modulus of the wavelet transform, the energy density of the wavelet transform, or any combination of the foregoing signals. For example, the data modeling processor may recognize the pulse band in a scalogram derived from a continuous wavelet transform of a PPG signal prior to corruption by artifact. The data modeling processor may then detect an unrecognizable (or low fidelity) pulse band during artifact corruption. Signal segments may then be gated for use only when the pulse band exceeds some predefined signal integrity threshold.

In an embodiment, the data modeling processor may learn signal characteristics associated with a particular physiological parameter to be determined using a supervised learning phase (e.g., a feed-forward multilayered network may use a gradient decent paradigm to minimize a system cost function). In an embodiment, the data modeling processor may implement a self-organizing map (SOM) feature (e.g., using a Kohonen map) that is trained using an unsupervised learning phase. A reinforcement learning phase (e.g., one that discovers a policy that minimizes some long-term cost metric) may additionally or alternatively be employed.

In an embodiment, the data modeling processor may implement a recurrent artificial neural network (e.g., a Hopfield network). The recurrent artificial neural network may converge on a stable solution (e.g., the noise-free version of the input). From the stable solution of the recurrent artificial neural network, regions of artifact may be detected. Valid signal segments may then be identified and may include segments not identified as having artifact (or having less than some threshold level of artifact), segments that are not stale (e.g., segments collected within some user-defined freshness threshold), or segments that are both free from artifact and not stale. The valid signal segments may then be used to determine one or more physiological parameters while the invalid signal segments are discarded or removed. One or more previously valid physiological parameter measurements may be held or buffered until a new valid measurement is determined from the valid signal segments.

In an embodiment, physiological parameters may be outputted in real-time using the data modeling processor. When a requisite length of a valid signal segment is received, a new physiological parameter measurement may be taken and outputted (e.g., displayed). If a region of invalid signal segments is encountered, previously known-good physiological measurements may be held until a sufficient valid signal segment is received and used to determine an updated physiological measurement. In an embodiment, an alarm (e.g., audible or visual alarm) may be automatically triggered when a measurement is stale (e.g., derived from signals received beyond some elapsed threshold time window).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above and other features of the present disclosure, its nature and various advantages will be more apparent upon consideration of the following detailed description, taken in conjunction with the accompanying drawings in which:

FIG. 1 shows an illustrative pulse oximetry system in accordance with an embodiment;

FIG. 2 is a block diagram of the illustrative pulse oximetry system of FIG. 1 coupled to a patient in accordance with an embodiment;

FIGS. 3(a) and 3(b) show illustrative views of a scalogram derived from a PPG signal in accordance with an embodiment;

FIG. 3(c) shows an illustrative scalogram derived from a signal containing two pertinent components in accordance with an embodiment;

FIG. 3(d) shows an illustrative schematic of signals associated with a ridge in FIG. 3(c) and illustrative schematics of a further wavelet decomposition of these newly derived signals in accordance with an embodiment;

FIGS. 3(e) and 3(f) are flow charts of illustrative steps involved in performing an inverse continuous wavelet transform in accordance with some embodiments;

FIG. 4 is a block diagram of an illustrative continuous wavelet processing system in accordance with some embodiments;

FIG. 5 shows an illustrative scalogram of a red PPG signal with an artifact present in accordance with an embodiment;

FIG. 6 shows an illustrative wavelet ratio surface derived from red and infrared PPG signals in accordance with an embodiment; and

FIGS. 7(a), 7(b), and 8 show illustrative processes for determining at least one physiological parameter in accordance with some embodiments.

### DETAILED DESCRIPTION

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient) and changes in blood volume in the skin. Ancillary to the blood oxygen saturation measurement, pulse oximeters may also be used to measure the pulse rate of the patient. Pulse oximeters typically measure and display various blood flow characteristics including, but not limited to, the oxygen saturation of hemoglobin in arterial blood.

An oximeter may include a light sensor that is placed at a site on a patient, typically a fingertip, toe, forehead or earlobe, or in the case of a neonate, across a foot. The oximeter may pass light using a light source through blood perfused tissue and photoelectrically sense the absorption alight in the tissue. For example, the oximeter may measure the intensity of light that is received at the light sensor as a function of time. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate the amount of the blood constituent (e.g., oxyhemoglobin) being measured as well as the pulse rate and when each individual pulse occurs.

The light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption, Red and infrared wavelengths may be used because it has been observed that highly oxygenated blood will absorb relatively less red light and more infrared light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based on Lambert-Beefs law. The following notation will be used herein:

I(λ,t)=Iₒ(λ)exp(−(sβₒ(λ)+(1−s)βᵣ(λ))l(t)) (1)

where:
λ=wavelength;
t=time;
I=intensity of light detected;
Iₒ=intensity of light transmitted;
s=oxygen saturation;
βₒ,βᵣ=empirically derived absorption coefficients; and
l(t)=a combination of concentration and path length from emitter to detector as a function of time.

The traditional approach measures light absorption at two wavelengths (e.g., red and infrared (IR)), and then calculates saturation by solving for the “ratio of ratios” as follows.
1. First, the natural logarithm of (1) is taken (“log” will be used to represent the natural logarithm) for IR and Red

log I=log Iₒ−(sβₒ+(1−s)βᵣ)l (2)
2. (2) is then differentiated with respect to time

$$\frac{d log I}{d t} = - ( s {β}_{o} + ( 1 - s ) {β}_{r} ) \frac{d l}{d t} \qquad ( 3 )$$

3. Red (3) is divided by IR (3)

$$\frac{d log I ( {λ}_{R} ) / d t}{d log I ( {λ}_{IR} ) / d t} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} \qquad ( 4 )$$

4. Solving for s

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\begin{matrix}\frac{d log I ( {λ}_{R} )}{d t} ( {β}_{o} ( {λ}_{IR} ) - {β}_{r} ( {λ}_{IR} ) ) - \\ \frac{d log I ( {λ}_{IR} )}{d t} ( {β}_{o} ( {λ}_{R} ) - {β}_{r} ( {λ}_{R} ) )\end{matrix}}$$

Note in discrete time

$$\frac{d log I ( λ , t )}{d t} ≃ log I ( λ , {t}_{2} ) - log I ( λ , {t}_{1} )$$

### Using log A-log B=log A/B,

$$\frac{d log I ( λ , t )}{d t} ≃ log ( \frac{ I ( {t}_{2} , λ )}{I ( {t}_{1} , λ )} )$$

So, (4) can be rewritten as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R \qquad ( 5 )$$

where R represents the “ratio of ratios.” Solving (4) for s using (5) gives

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) + {β}_{r} ( {λ}_{R} )} .$$

From (5), R can be calculated using two points (e.g., PPG maximum and minimum), or a family of points. One method using a family of points uses a modified version of (5). Using the relationship

$$\frac{d log I}{d t} = \frac{d I / d t}{I} \qquad ( 6 )$$

now (5) becomes

$$\begin{matrix}\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} \\ = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} \\ = R\end{matrix} \qquad ( 7 )$$

which defines a cluster of points whose slope of y versus x will give R where

x(t)=[I(t₂,λ_{IR})−I(t₁,λ_{IR})]I(t₁,λ_{R})

y(t)=[I(t₂,λ_{R})−I(t₁,λ_{R})]I(t₁,λ_{IR})

y(t)=Rx(t) (8)

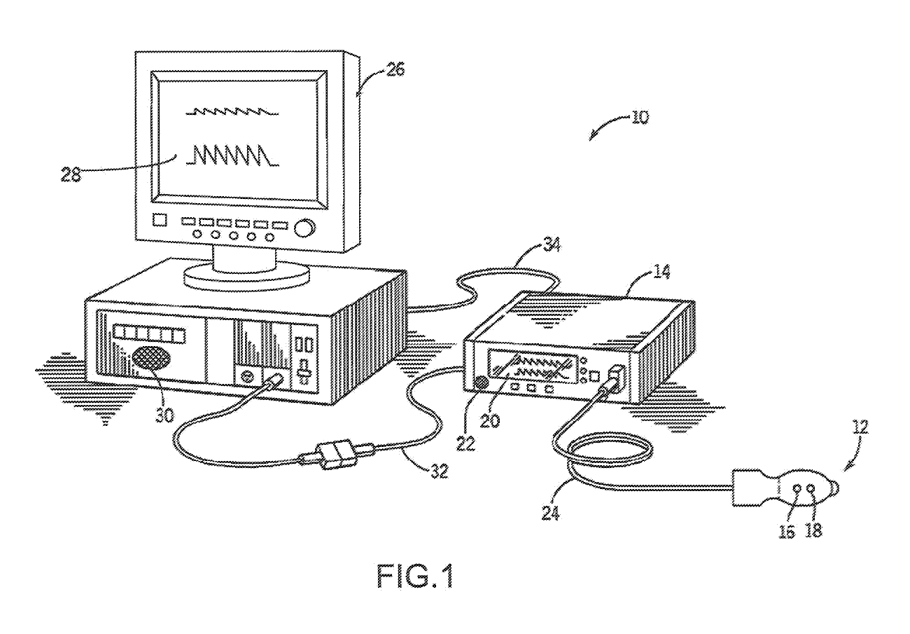

FIG. 1 is a perspective view of an embodiment of a pulse oximetry system 10. System 10 may include a sensor 12 and a pulse oximetry monitor 14. Sensor 12 may include an emitter 16 for emitting light at two or more wavelengths into a patient's tissue. A detector 18 may also be provided in sensor 12 for detecting the light originally from emitter 16 that emanates from the patient's tissue after passing through the tissue.

According to another embodiment and as will be described, system 10 may include a plurality of sensors forming a sensor array in lieu of single sensor 12. Each of the sensors of the sensor array may be a complementary metal oxide semiconductor (CMOS) sensor. Alternatively, each sensor of the array may be charged coupled device (CCD) sensor. In another embodiment, the sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier.

According to an embodiment, emitter 16 and detector 18 may be on opposite sides of a digit such as a finger or toe, in which case the light that is emanating from the tissue has passed completely through the digit. In an embodiment, emitter 16 and detector 18 may be arranged so that light from emitter 16 penetrates the tissue and is reflected by the tissue into detector 18, such as a sensor designed to obtain pulse oximetry data from a patient's forehead.

In an embodiment, the sensor or sensor array may be connected to and draw its power from monitor 14 as shown. In another embodiment, the sensor may be wirelessly connected to monitor 14 and include its own battery or similar power supply (not shown). Monitor 14 may be configured to calculate physiological parameters based at least in part on data received from sensor 12 relating to light emission and detection. In an alternative embodiment, the calculations may be performed on the monitoring device itself and the result of the oximetry reading may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range.

In an embodiment, sensor 12, or the sensor array, may be communicatively coupled to monitor 14 via a cable 24. However, in other embodiments, a wireless transmission device (not shown) or the like may be used instead of or in addition to cable 24.

In the illustrated embodiment, pulse oximetry system 10 may also include a multi-parameter patient monitor 26. The monitor may be cathode ray tube type, a flat panel display (as shown) such as a liquid crystal display (LCD) or a plasma display, or any other type of monitor now known or later developed. Multi-parameter patient monitor 26 may be configured to calculate physiological parameters and to provide a display 28 for information from monitor 14 and from other medical monitoring devices or systems (not shown). For example, multiparameter patient monitor 26 may be configured to display an estimate of a patient's blood oxygen saturation generated by pulse oximetry monitor 14 (referred to as an “SpO₂” measurement), pulse rate information from monitor 14 and blood pressure from a blood pressure monitor (not shown) on display 28.

Monitor 14 may be communicatively coupled to multi-parameter patient monitor 26 via a cable 32 or 34 that is coupled to a sensor input port or a digital communications port, respectively and/or may communicate wirelessly (not shown). In addition, monitor 14 and/or multi-parameter patient monitor 26 may be coupled to a network to enable the sharing of information with servers or other workstations (not shown). Monitor 14 may be powered by a battery (not shown) or by a conventional power source such as a wall outlet.

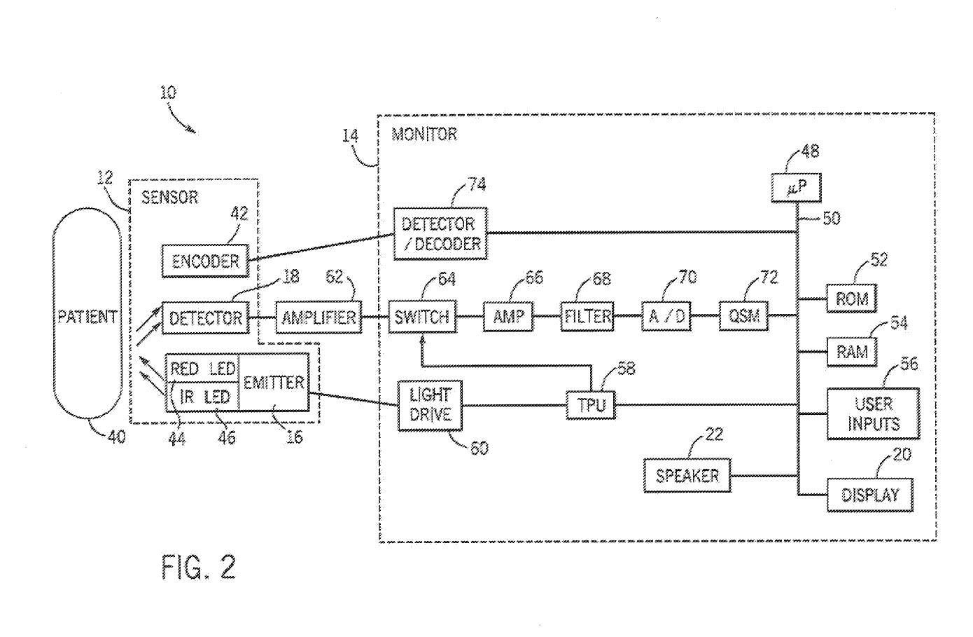

FIG. 2 is a block diagram of a pulse oximetry system, such as pulse oximetry system 10 of FIG. 1, which may be coupled to a patient 40 in accordance with an embodiment. Certain illustrative components of sensor 12 and monitor 14 are illustrated in FIG. 2. Sensor 12 may include emitter 16, detector 18, and encoder 42. In the embodiment shown, emitter 16 may be configured to emit at least two wavelengths of light (e.g., RED and IR) into a patient's tissue 40. Hence, emitter 16 may include a RED light emitting light source such as RED light emitting diode (LED) 44 and an IR light emitting light source such as IR LED 46 for emitting light into the patient's tissue 40 at the wavelengths used to calculate the patient's physiological parameters. In one embodiment, the RED wavelength may be between about 600 nm and about 700 nm, and the IR wavelength may be between about 800 nm and about 1000 nm. In embodiments where a sensor array is used in place of single sensor, each sensor may be configured to emit a single wavelength. For example, a first sensor emits only a RED light while a second only emits an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In an embodiment, detector 18 may be configured to detect the intensity of light at the RED and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the RED and IR wavelengths in the patient's tissue 40.

In an embodiment, encoder 42 may contain information about sensor 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patients physiological parameters.

Encoder 42 may contain information specific to patient 40, such as, for example, the patient's age, weight, and diagnosis. This information may allow monitor 14 to determine, for example, patient-specific threshold ranges in which the patient's physiological parameter measurements should fall and to enable or disable additional physiological parameter algorithms. Encoder 42 may, for instance, be a coded resistor which stores values corresponding to the type of sensor 12 or the type of each sensor in the sensor array, the wavelengths of light emitted by emitter 16 on each sensor of the sensor array, and/or the patients characteristics. In another embodiment, encoder 42 may include a memory on which one or more of the following information may be stored for communication to monitor 14: the type of the sensor 12; the wavelengths of light emitted by emitter 16; the particular wavelength each sensor in the sensor array is monitoring; a signal threshold for each sensor in the sensor array; any other suitable information; or any combination thereof.

In an embodiment, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 58 may provide timing control signals to a light drive circuitry 60, which may control when emitter 16 is illuminated and multiplexed timing for the RED LED 44 and the IR LED 46. TPU 58 may also control the gating-in of signals from detector 18 through an amplifier 62 and a switching circuit 64. These signals are sampled at the proper time, depending upon which light source is illuminated. The received signal from detector 18 may be passed through an amplifier 66, a low pass filter 68, and an analog-to-digital converter 70. The digital data may then be stored in a queued serial module (QSM) 72 (or buffer) for later downloading to RAM 54 as QSM 72 fills up. In one embodiment, there may be multiple separate parallel paths having amplifier 66, filter 68, and A/D converter 70 for multiple light wavelengths or spectra received.

In an embodiment, microprocessor 48 may determine the patient's physiological parameters, such as SpO₂ and pulse rate, using various algorithms and/or look-up tables based on the value of the received signals and/or data corresponding to the light received by detector 18. Signals corresponding to information about patient 40, and particularly about the intensity of light emanating from a patient's tissue over time, may be transmitted from encoder 42 to a decoder 74. These signals may include, for example, encoded information relating to patient characteristics. Decoder 74 may translate these signals to enable the microprocessor to determine the thresholds based on algorithms or look-up tables stored in ROM 52. User inputs 56 may be used to enter information about the patient, such as age, weight, height, diagnosis, medications, treatments, and so forth. In an embodiment, display 20 may exhibit a list of values which may generally apply to the patient, such as, for example, age ranges or medication families, which the user may select using user inputs 56.

In an embodiment, microprocessor 48 may include (or be in communication with or coupled to) a data modeling processor. The data modeling processor may include memory (e.g., RAM, ROM, and hybrid types of memory), graphics circuitry (not shown), and digital signal processing (DSP) circuitry coupled to the memory and graphics circuitry. As described in more detail below, in some embodiments, the data modeling processor may implement an artificial neural network to identify patterns and characteristic features in the received signals and/or data corresponding to the light received by detector 18. The data modeling processor may take as an input a PPG signal, a transformed version of a PPG signal, a scalogram of the transformed version of a PPG signal, or any other wavelet representation of the received signals and/or data corresponding to the light received by detector 18. The data modeling processor may additionally or alternatively take as an input a parameterized version of any of the foregoing signals or signal representations, as discussed in more detail below. The data modeling processor may identify or gate signal segments that may be used to determine physiological parameters while ignoring, weighting to zero, or replacing signal segments that contain artifact. In an embodiment, the signal segments that contain artifact may be replaced with previously received artifact-free signal segments (e.g., signal segments received immediately prior to the segments determined to contain artifact). In this way, only non-corrupted signal segments may be used in determining physiological parameters.

The optical signal through the tissue can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. In addition, because blood is a fluid, it responds differently than the surrounding tissue to inertial effects, thus resulting in momentary changes in volume at the point to which the oximeter probe is attached.

Noise (e.g., from patient movement) can degrade a pulse oximetry signal relied upon by a physician, without the physician's awareness. This is especially true if the monitoring of the patient is remote, the motion is too small to be observed, or the doctor is watching the instrument or other parts of the patient, and not the sensor site. Processing pulse oximetry (i.e., PPG) signals may involve operations that reduce the amount of noise present in the signals or otherwise identify noise components in order to prevent them from affecting measurements of physiological parameters derived from the PPG signals.

It will be understood that the present disclosure is applicable to any suitable signals and that PPG signals are used merely for illustrative purposes. Those skilled in the art will recognize that the present disclosure has wide applicability to other signals including, but not limited to other biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In one embodiment, a PPG signal may be transformed using a continuous wavelet transform. Information derived from the transform of the PPG signal (i.e., in wavelet space) may be used to provide measurements gone or more physiological parameters.

The continuous wavelet transform of a signal x(t) in accordance with the present disclosure may be defined as

$$T ( a , b ) = \frac{1}{\sqrt{a}} {\int }_{- ∞}^{+ ∞} x ( t ) {ψ}^{*} ( \frac{t - b}{a} ) d t \qquad ( 9 )$$

where ψ*(t) is the complex conjugate of the wavelet function ψ(t), a is the dilation parameter of the wavelet and b is the location parameter of the wavelet. The transform given by equation (9) may be used to construct a representation of a signal on a transform surface. The transform may be regarded as a time-scale representation. Wavelets are composed of a range of frequencies, one of which may be denoted as the characteristic frequency of the wavelet, where the characteristic frequency associated with the wavelet is inversely proportional to the scale a. One example of a characteristic frequency is the dominant frequency. Each scale of a particular wavelet may have a different characteristic frequency. The underlying mathematical detail required for the implementation within a time-scale can be found, for example, in Paul S. Addison, The Illustrated Wavelet Transform Handbook (Taylor & Francis Group 2002), which is hereby incorporated by reference herein in its entirety.

The continuous wavelet transform decomposes a signal using wavelets, which are generally highly localized in time. The continuous wavelet transform may provide a higher resolution relative to discrete transforms, thus providing the ability to garner more information from signals than typical frequency transforms such as Fourier transforms (or any other spectral techniques) or discrete wavelet transforms. Continuous wavelet transforms allow for the use of a range of wavelets with scales spanning the scales of interest of a signal such that small scale signal components correlate well with the smaller scale wavelets and thus manifest at high energies at smaller scales in the transform. Likewise, large scale signal components correlate well with the larger scale wavelets and thus manifest at high energies at larger scales in the transform. Thus, components at different scales may be separated and extracted in the wavelet transform domain. Moreover, the use of a continuous range of wavelets in scale and time position allows for a higher resolution transform than is possible relative to discrete techniques.

In addition, transforms and operations that convert a signal or any other type of data into a spectral (i.e., frequency) domain necessarily create a series of frequency transform values in a two-dimensional coordinate system where the two dimensions may be frequency and, for example, amplitude. For example, any type of Fourier transform would generate such a two-dimensional spectrum. In contrast, wavelet transforms, such as continuous wavelet transforms, are required to be defined in a three-dimensional coordinate system and generate a surface with dimensions of time, scale and, for example, amplitude. Hence, operations performed in a spectral domain cannot be performed in the wavelet domain; instead the wavelet surface must be transformed into a spectrum (L e., by performing an inverse wavelet transform to convert the wavelet surface into the time domain and then performing a spectral transform from the time domain). Conversely, operations performed in the wavelet domain cannot be performed in the spectral domain; instead a spectrum must first be transformed into a wavelet surface (i.e., by performing an inverse spectral transform to convert the spectral domain into the time domain and then performing a wavelet transform from the time domain). Nor does a cross-section of the three-dimensional wavelet surface along, for example, a particular point in time equate to a frequency spectrum upon which spectral-based techniques may be used. At least because wavelet space includes a time dimension, spectral techniques and wavelet techniques are not interchangeable. It will be understood that converting a system that relies on spectral domain processing to one that relies on wavelet space processing would require significant and fundamental modifications to the system in order to accommodate the wavelet space processing (e.g., to derive a representative energy value for a signal or part of a signal requires integrating twice, across time and scale, in the wavelet domain while, conversely, one integration across frequency is required to derive a representative energy value from a spectral domain). As a further example, to reconstruct a temporal signal requires integrating twice, across time and scale, in the wavelet domain while, conversely, one integration across frequency is required to derive a temporal signal from a spectral domain. It is well known in the art that, in addition to or as an alternative to amplitude, parameters such as energy density, modulus, phase, among others may all be generated using such transforms and that these parameters have distinctly different contexts and meanings when defined in a two-dimensional frequency coordinate system rather than a three-dimensional wavelet coordinate system. For example, the phase of a Fourier system is calculated with respect to a single origin for all frequencies while the phase for a wavelet system is unfolded into two dimensions with respect to a wavelet's location (often in time) and scale.

The energy density function of the wavelet transform, the scalogram, is defined as

S(a,b)=|T(a,b)|² (10)

where ‘∥’ is the modulus operator. The scalogram may be resealed for useful purposes. One common resealing is defined as

$${S}_{R} ( a , b ) = \frac{{\vert T ( a , b ) \vert}^{2}}{a} \qquad ( 11 )$$

and is useful for defining ridges in wavelet space when, for example, the Morlet wavelet is used. Ridges are defined as the locus of points of local maxima in the plane. Any reasonable definition of a ridge may be employed in the method. Also included as a definition of a ridge herein are paths displaced from the locus of the local maxima. A ridge associated with only the locus of points of local maxima in the plane are labeled a “maxima ridge”.

For implementations requiring fast numerical computation, the wavelet transform may be expressed as an approximation using Fourier transforms. Pursuant to the convolution theorem, because the wavelet transform is the cross-correlation of the signal with the wavelet function, the wavelet transform may be approximated in terms of an inverse FFT of the product of the Fourier transform of the signal and the Fourier transform of the wavelet for each required a scale and then multiplying the result by \sqrt{a}.

In the discussion of the technology which follows herein, the “scalogram” may be taken to include all suitable forms of resealing including, but not limited to, the original unsealed wavelet representation, linear resealing, any power of the modulus of the wavelet transform, or any other suitable resealing. In addition, for purposes of clarity and conciseness, the term “scalogram” shall be taken to mean the wavelet transform, T(a,b) itself, or any part thereof. For example, the real part of the wavelet transform, the imaginary part of the wavelet transform, the phase of the wavelet transform, any other suitable part of the wavelet transform, or any combination thereof is intended to be conveyed by the term “scalogram”.

A scale, which may be interpreted as a representative temporal period, may be converted to a characteristic frequency of the wavelet function. The characteristic frequency associated with a wavelet of arbitrary a scale is given by

$$f = \frac{{f}_{c}}{a} \qquad ( 12 )$$

where f_{c}, the characteristic frequency of the mother wavelet (i.e., at a=1), becomes a scaling constant and f is the representative or characteristic frequency for the wavelet at arbitrary scale a.

Any suitable wavelet function may be used in connection with the present disclosure. One of the most commonly used complex wavelets, the Morlet wavelet, is defined as:

ψ(t)=π^{-1/4}(e^{i2πf}^{₀}^{t}−e^{-2πf}^{₀}⁾^{²}^{/2})e^{-t}^{²}^{/2} (13)

where f₀ is the central frequency of the mother wavelet. The second term in the parenthesis is known as the correction term, as it corrects for the non-zero mean of the complex sinusoid within the Gaussian window. In practice, it becomes negligible for values of f₀>>0 and can be ignored, in which case, the Morlet wavelet can be written in a simpler form as

$$ψ ( t ) = \frac{1}{{π}^{1 / 4}} {e}^{i2π {f}_{0} t} {e}^{- {i}^{2} / 2} \qquad ( 14 )$$

This wavelet is a complex wave within a scaled Gaussian envelope. While both definitions of the Morlet wavelet are included herein, the function of equation (14) is not strictly a wavelet as it has a non-zero mean (i.e., the zero frequency term of its corresponding energy spectrum is non-zero). However, it will be recognized by those skilled in the art that equation (14) may be used in practice with f₀>>0 with minimal error and is included (as well as other similar near wavelet functions) in the definition of a wavelet herein. A more detailed overview of the underlying wavelet theory, including the definition of a wavelet function, can be found in the general literature. Discussed herein is how wavelet transform features may be extracted from the wavelet decomposition of signals. For example, wavelet decomposition of PPG signals may be used to provide clinically useful information within a medical device.

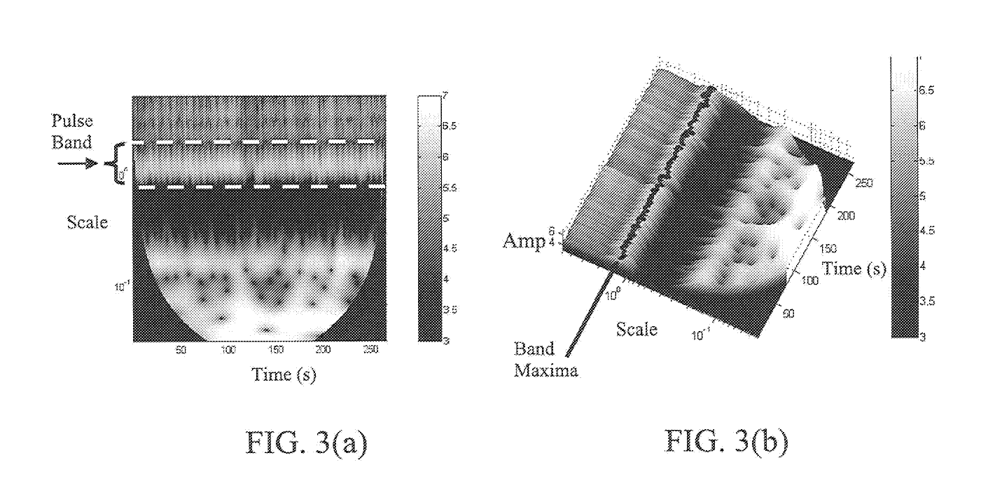

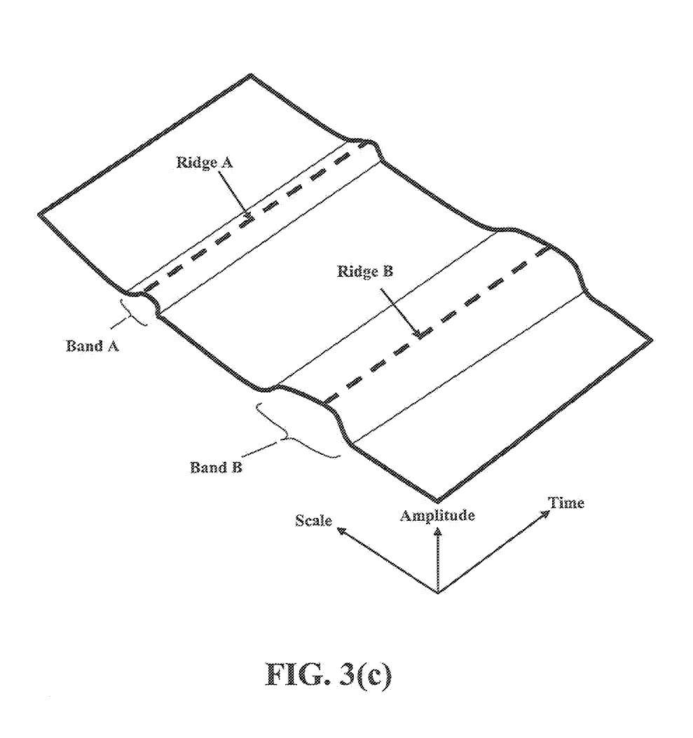

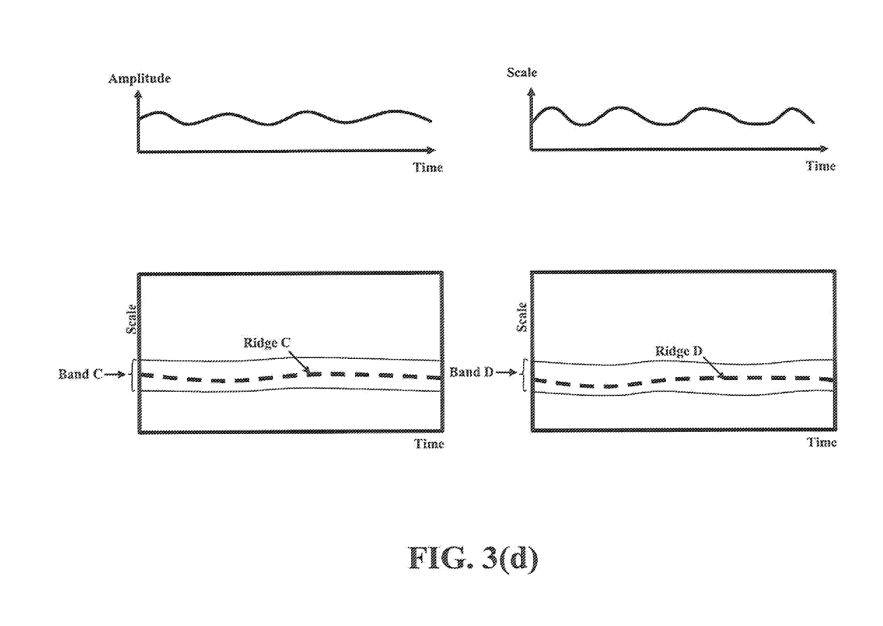

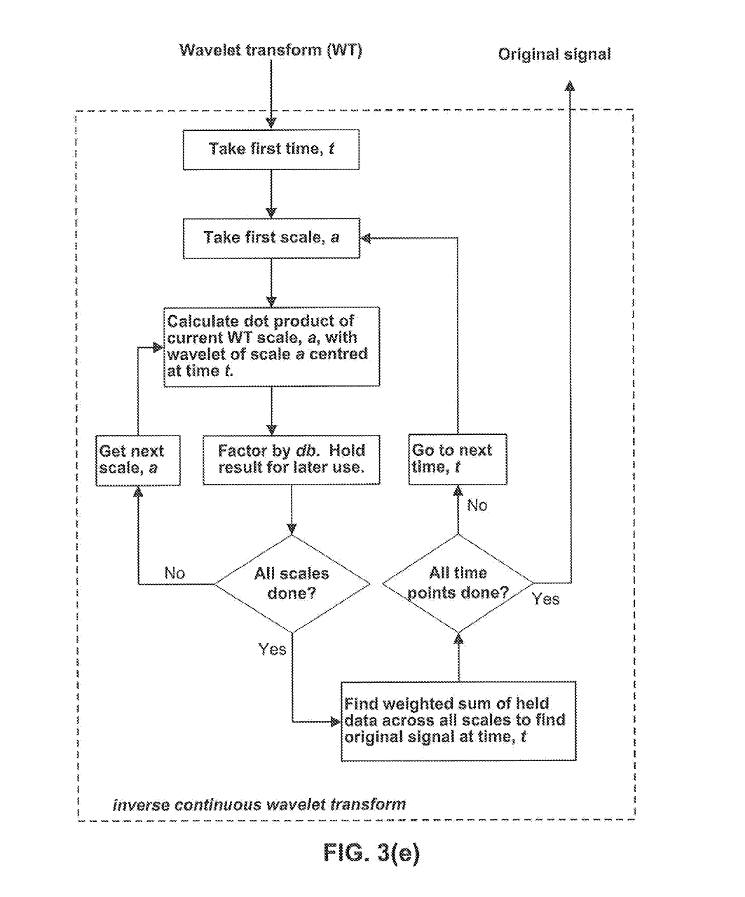

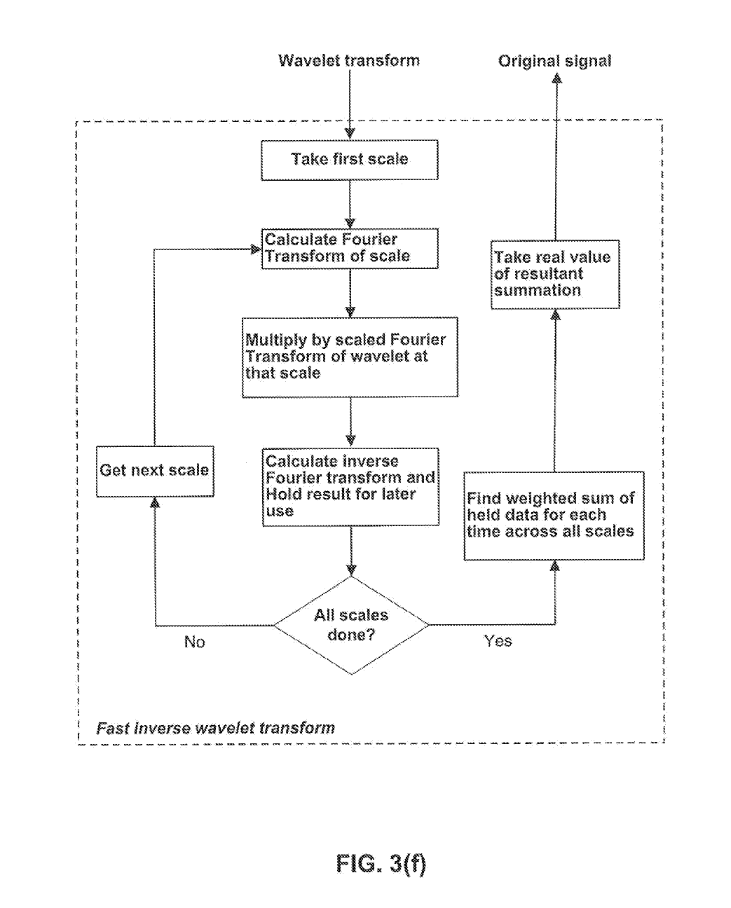

Pertinent repeating features in a signal give rise to a time-scale band in wavelet space or a resealed wavelet space. For example, the pulse component of a PPG signal produces a dominant band in wavelet space at or around the pulse frequency. FIGS. 3(a) and (b) show two views of an illustrative scalogram derived from a PPG signal, according to an embodiment. The figures show an example of the band caused by the pulse component in such a signal. The pulse band is located between the dashed lines in the plot of FIG. 3(a). The band is formed from a series of dominant coalescing features across the scalogram. This can be clearly seen as a raised band across the transform surface in FIG. 3(b) located within the region of scales indicated by the arrow in the plot (corresponding to 60 beats per minute). The maxima of this band with respect to scale is the ridge. The locus of the ridge is shown as a black curve on top of the band in FIG. 3(b). By employing a suitable resealing of the scalogram, such as that given in equation (11), the ridges found in wavelet space may be related to the instantaneous characteristic frequency of the signal. In this way, the pulse rate may be obtained from the PPG signal. Instead of resealing the scalogram, a suitable predefined relationship between the scale obtained from the ridge on the wavelet surface and the actual pulse rate may also be used to determine the pulse rate.

By mapping the time-scale coordinates of the pulse ridge onto the wavelet phase information gained through the wavelet transform, individual pulses may be captured. In this way, both times between individual pulses and the timing of components within each pulse may be monitored and used to detect heart beat anomalies, measure arterial system compliance, or perform any other suitable calculations or diagnostics. Alternative definitions of a ridge may be employed. Alternative relationships between the ridge and the pulse frequency of occurrence may be employed.

As discussed above, pertinent repeating features in the signal give rise to a time-scale band in wavelet space or a resealed wavelet space. For a periodic signal, this band remains at a constant scale in the time-scale plane. For many real signals, especially biological signals, the band may be non-stationary; varying in scale, amplitude, or both over time. FIG. 3(c) shows an illustrative schematic of a wavelet transform of a signal containing two pertinent components leading to two bands in the transform space, according to an embodiment. These bands are labeled band A and band B on the three-dimensional schematic of the wavelet surface. In this embodiment, the band ridge is defined as the locus of the peak values of these bands with respect to scale. For purposes of discussion, it may be assumed that band B contains the signal information of interest. This will be referred to as the “primary band”. In addition, it may be assumed that the system from which the signal originates, and from which the transform is subsequently derived, exhibits some form of coupling between the signal components in band A and band B. When noise or other erroneous features are present in the signal with similar spectral characteristics of the features of band B then the information within band B can become ambiguous (i.e., obscured, fragmented or missing). In this case, the ridge of band A may be followed in wavelet space and extracted either as an amplitude signal or a scale signal which will be referred to as the “ridge amplitude perturbation” (RAP) signal and the “ridge scale perturbation” (RSP) signal, respectively. The RAP and RSP signals may be extracted by projecting the ridge onto the time-amplitude or time-scale planes, respectively. The top plots of FIG. 3(d) show a schematic of the RAP and RSP signals associated with ridge A in FIG. 3(c). Below these RAP and RSP signals are schematics of a further wavelet decomposition of these newly derived signals. This secondary wavelet decomposition allows for information in the region of band B in FIG. 3(c) to be made available as band C and band D. The ridges of bands C and D may serve as instantaneous time-scale characteristic measures of the signal components causing bands C and D. This technique, which will be referred to herein as secondary wavelet feature decoupling (SWFD), may allow information concerning the nature of the signal components associated with the underlying physical process causing the primary band B (FIG. 3(c)) to be extracted when band B itself is obscured in the presence of noise or other erroneous signal features.

In some instances, an inverse continuous wavelet transform may be desired, such as when modifications to a scalogram (or modifications to the coefficients of a transformed signal) have been made in order to, for example, remove artifacts. In one embodiment, there is an inverse continuous wavelet transform which allows the original signal to be recovered from its wavelet transform by integrating over all scales and locations, a and b:

$$x ( t ) = \frac{1}{{C}_{g}} {\int }_{- ∞}^{∞} {\int }_{0}^{∞} T ( a , b ) \frac{1}{\sqrt{a}} ψ ( \frac{t - b}{a} ) \frac{d a d b}{{a}^{2}} \qquad ( 15 )$$

which may also be written as:

$$x ( t ) = \frac{1}{{C}_{g}} {\int }_{- ∞}^{∞} {\int }_{0}^{∞} T ( a , b ) {ψ}_{a , b} ( t ) \frac{d a d b}{{a}^{2}} \qquad ( 16 )$$

where C_{g} is a scalar value known as the admissibility constant. It is wavelet type dependent and may be calculated from:

$${C}_{g} = {\int }_{o}^{∞} \frac{{\vert \hat{ψ} ( f ) \vert}^{2}}{f} d f \qquad ( 17 )$$

FIG. 3(e) is a flow chart of illustrative steps that may be taken to perform an inverse continuous wavelet transform in accordance with the above discussion. An approximation to the inverse transform may be made by considering equation (15) to be a series of convolutions across scales. It shall be understood that there is no complex conjugate here, unlike for the cross correlations of the forward transform. As well as integrating over all of a and b for each time t, this equation may also take advantage of the convolution theorem which allows the inverse wavelet transform to be executed using a series of multiplications. FIG. 3(f) is a flow chart of illustrative steps that may be taken to perform an approximation of an inverse continuous wavelet transform. It will be understood that any other suitable technique for performing an inverse continuous wavelet transform may be used in accordance with the present disclosure.

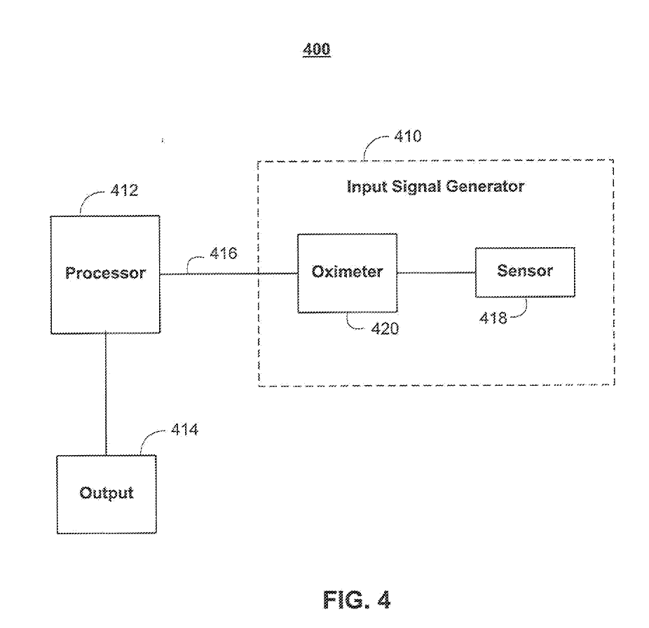

FIG. 4 is an illustrative continuous wavelet processing system in accordance with an embodiment. In this embodiment, input signal generator 410 generates an input signal 416. As illustrated, input signal generator 410 may include oximeter 420 coupled to sensor 418, which may provide as input signal 416, a PPG signal. It will be understood that input signal generator 410 may include any suitable signal source, signal generating data, signal generating equipment, or any combination thereof to produce signal 416. Signal 416 may be any suitable signal or signals, such as, for example, biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In this embodiment, signal 416 may be coupled to processor 412. Processor 412 may be any suitable software, firmware, and/or hardware, and/or combinations thereof for processing signal 416. For example, processor 412 may include one or more hardware processors (e.g., integrated circuits), one or more software modules, computer-readable media such as memory, firmware, or any combination thereof. Processor 412 may, for example, be a computer or may be one or more chips (i.e., integrated circuits). Processor 412 may perform the calculations associated with the continuous wavelet transforms of the present disclosure as well as the calculations associated with any suitable interrogations of the transforms. Processor 412 may perform any suitable signal processing of signal 416 to filter signal 416, such as any suitable band-pass filtering, adaptive filtering, closed-loop filtering, and/or any other suitable filtering, and/or any combination thereof.

Processor 412 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 412 to, for example, store data corresponding to a continuous wavelet transform of input signal 416, such as data representing a scalogram. In one embodiment, data representing a scalogram may be stored in RAM or memory internal to processor 412 as any suitable three-dimensional data structure such as a three-dimensional array that represents the scalogram as energy levels in a time-scale plane. Any other suitable data structure may be used to store data representing a scalogram.

Processor 412 may be coupled to output 414. Output 414 may be any suitable output device such as, for example, one or more medical devices (e.g., a medical monitor that displays various physiological parameters, a medical alarm, or any other suitable medical device that either displays physiological parameters or uses the output of processor 412 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 400 may be incorporated into system 10 (FIGS. 1 and 2) in which, for example, input signal generator 410 may be implemented as parts of sensor 12 and monitor 14 and processor 412 may be implemented as part of monitor 14.

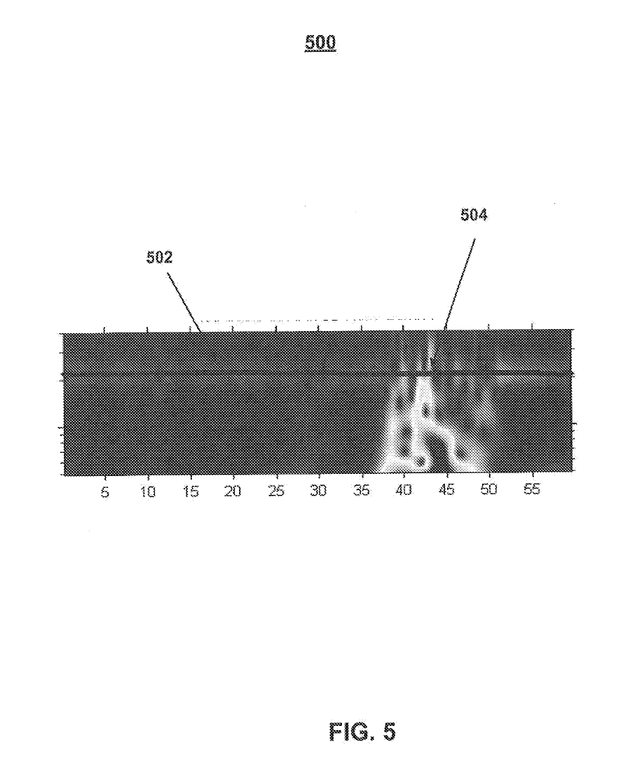

FIG. 5 shows illustrative scalogram 500. Although, in the depicted embodiment, scalogram 500 is derived from a red PPG signal, scalogram 500 could be derived from any suitable signal detected from any suitable energy source (e.g., a light source) at any frequency (e.g., infrared) and intensity. As described above, pertinent repeating features in a signal may give rise to a time-scale band in wavelet space or a resealed wavelet space. As shown in FIG. 5, scalogram 500 includes such a band at pulse band 502. Artifact region 504 can be seen across scalogram 500 at around 40 to 50 seconds. Artifact region 504 may corrupt pulse band 502, making it less discernible in scalogram 500. Because the determination of some physiological parameters (e.g., pulse rate) may depend, at least in part, on the proper identification of pulse band 502, artifact region 504 may result in inaccurate physiological measurements. For example, artifact region 504 may cause a physiological monitoring system (e.g., a pulse oximetry system) that derives a pulse rate or SpO₂ value at least in part from scalogram 500 to output a skewed or corrupted measurement after processing artifact region 504.

In order to identify non-corrupted signal segments in scalogram 500 (or any other wavelet representation of an underlying detected signal), a data modeling processor may be employed (e.g., in microprocessor 48 (FIG. 2)). In an embodiment, regions free from artifact may be identified and flagged (or “gated”) for use in determining physiological parameters, such as oxygen saturation, pulse rate, respiration rate, respiratory effort, and blood pressure. The artifact-free regions may be identified or gated in real-time as the underlying signal is collected (e.g., from a pulse oximetry system). Real-time identification of non-corrupt or artifact-free scalogram segments may allow for continuous output of a patient's physiological parameters derived, at least in part, from those non-corrupt or artifact-free segments. Previously known values of the patient's physiological parameters may be buffered until a suitable artifact-free region is detected for an updated valid measurement.

As shown in FIG. 5, the data modeling processor may recognize that artifact region 504 is corrupting pulse band 502 from 40 to 50 seconds on the x-axis of scalogram 500. In response to detecting this corruption of pulse band 502, the data modeling processor may stop gating the corrupted signal segment (e.g., from 40 to 50 seconds) for use in determining physiological parameters. In some embodiments, the entire signal (e.g., a two-dimensional slice of scalogram 500) is flagged as corrupted and not used in determining physiological parameters. In other embodiments, only the pertinent portions of the signal are flagged as corrupted and not used in determining physiological parameters. For example, pulse band 502 may be the pertinent portion of scalogram 500 used in determining pulse rate. For other physiological parameters, other portions of scalogram 500 (e.g., the breathing band or the entire scalogram) may be the pertinent portions.

In an embodiment, when an artifact is detected that corrupts a pertinent portion of scalogram 500, the previous values in the scalogram may be held until the data modeling processor recognizes another non-corrupted signal segment. In some embodiments, the corrupted signal segments may be removed or weighted to zero when determining physiological parameters. In other embodiments, the corrupted signal segments may be replaced with previously known-good values or expected values. For example, the data modeling processor may use linear or nonlinear regression to extrapolate expected signal segments that best fit a known model. The extrapolated data may then replace the corresponding data in scalogram 500 containing artifact.

In an embodiment, a data modeling processor includes a non-linear statistical data modeling module that identifies valid scalogram segments for use in determining physiological parameters. The modeling processor (which may take the form of an artificial neural network in some embodiments) may be trained to identify scalogram segments that are valid for use in determining physiological parameters. Valid signal segments may then be identified and may include segments not identified as having artifact (or having less than some threshold level of artifact), segments that are not stale (e.g., segments collected within some user-defined freshness threshold), or segments that are both free from artifact and not stale. The valid signal segments may then be used to determine one or more physiological parameters while the invalid signal segments are discarded or removed from the scalogram (e.g., the invalid signal segments may be weighted to zero).

In an embodiment, the data modeling processor may operate directly on the detected signal itself (e.g., a PPG signal) or some transform of the detected signal (e.g., a continuous wavelet transform of a PPG signal). In some embodiments, the data modeling processor may also operate on a scalogram derived from the transformed signal, a wavelet ratio surface, the real part of the wavelet transform, the imaginary part of the wavelet transform, the modulus of the wavelet transform, the energy density of the wavelet transform, or any combination of the foregoing signals. For example, the data modeling processor may recognize the pulse band in a scalogram derived from a continuous wavelet transform of a PPG signal prior to corruption by artifact. The data modeling processor may then detect an unrecognizable pulse band during artifact corruption. Signal segments may then be gated for use only when the pulse band exceeds some predefined signal integrity threshold.

In an embodiment, the data modeling processor may learn signal characteristics associated with a particular physiological parameter to be determined using a supervised learning phase. A cost function (e.g., the mean squared error) may be defined, and the minimum cost may be determined using a first-order optimization algorithm (e.g., gradient descent). Any suitable backpropagation technique may be used for training the data modeling processor in supervised learning mode. Valid signal regions may then be identified and extracted for use in determining the physiological parameter based, at least in part, on the signal characteristics learned during the supervised learning phase. The data modeling processor may operate on the complete signal itself (or a transform of the complete signal) or some parameterized version of the signal (or some parameterized version of a transform of the signal).

In an embodiment, the data modeling processor may implement a self-organizing map (SOM) feature (e.g., using a Kohonen map) that is trained using an unsupervised learning phase. A cost function may be defined that depends on one or more a priori assumptions of the model used. In some embodiments, the cost function may be based, at least in part, on the posterior probability of the model given the input data. In some embodiments, the data modeling processor implements both a supervised learning phase and an unsupervised learning phase. A reinforcement learning phase (e.g., one that discovers a policy that minimizes some long-term cost metric) may additionally or alternatively be employed.

In an embodiment, the data modeling processor may implement a recurrent artificial neural network (e.g., a Hopfield network). Binary threshold units may be defined that take on two different states depending on whether the units' inputs exceed their threshold values. Each node in the network may move to a state that minimizes the energy associated with itself and its neighbors. The recurrent artificial neural network may then converge on a stable solution (e.g., the noise-free version of the input). From the stable solution of the recurrent artificial neural network, regions of artifact may then be detected. Valid signal segments may then be identified and may include segments not identified as having artifact (or having less than some threshold level of artifact), segments that are not stale (e.g., segments collected within some user-defined freshness threshold), or segments that are both free from artifact and not stale. The valid signal segments may then be used to determine one or more physiological parameters while the invalid signal segments are discarded or removed. One or more previously valid physiological parameter measurements may be held or buffered until a new valid measurement is determined from the valid signal segments.

In an embodiment, physiological parameters may be outputted using the data modeling processor in real-time. When a requisite length of a valid signal segment is received, a new physiological parameter measurement may be taken and outputted (e.g., displayed). If a region of invalid signal segments is encountered, previously known-good physiological measurements may be held until a sufficient valid signal segment is received and used to determine an updated physiological measurement. In an embodiment, an alarm (e.g., audible or visual alarm) may be automatically triggered when a measurement is stale (e.g., derived from signals received beyond some elapsed threshold time window).

In an embodiment, the neural network may take as input the scalogram magnitude values for scales about the expected pulse period. The network may be adapted to output a flag indicating artifact being present in the inputted data. During a training phase, the network's learning paradigm may calculate a cost function based on the difference between the flag value calculated for a given input and the target flag value for this input (for example, this data may have been collected with the presence of artifact recorded by another means, for example, by motion sensors). The weights of the network may then be altered in such a way as to reduce the error between the calculated output and target output of the network, for example, by means of gradient decent of the error surface or some other technique. Thus, in some embodiments, a training phase and operating phase may be defined as follows:

Training phase (supervised learning):

Start:
1. Collect new PPG section of training data;
2. Generate a scalogram including transformed newly collected data;
3. Select scalogram region for investigation;
4. Compress scalogram data for ANN presentation (optional);
5. Present data to ANN input units;
6. Calculate ANN output using rules of propagation and activation;
7. Compare ANN output flag with target output flag to determine a cost value;
8. Alter ANN weight matrices to reduce this cost value; and
9. iterate (return to start).

During the operating phase the network may, when presented with data similar to that on which it was trained, provide an output flag indicative of the presence of artifact. Thus,

Operating phase:

Start:
1. Collect new PPG data;
2. Generate a scalogram including transformed newly collected data;
3. Select scalogram region for investigation;
4. Compress scalogram data for ANN presentation (optional);
5. Present data to ANN input units;
6. Calculate ANN output flag using rules of propagation and activation;
7. Apply ANN output flag to gate the newly collected data's inclusion in physiological parameter derivation; and
8. iterate (return to start).

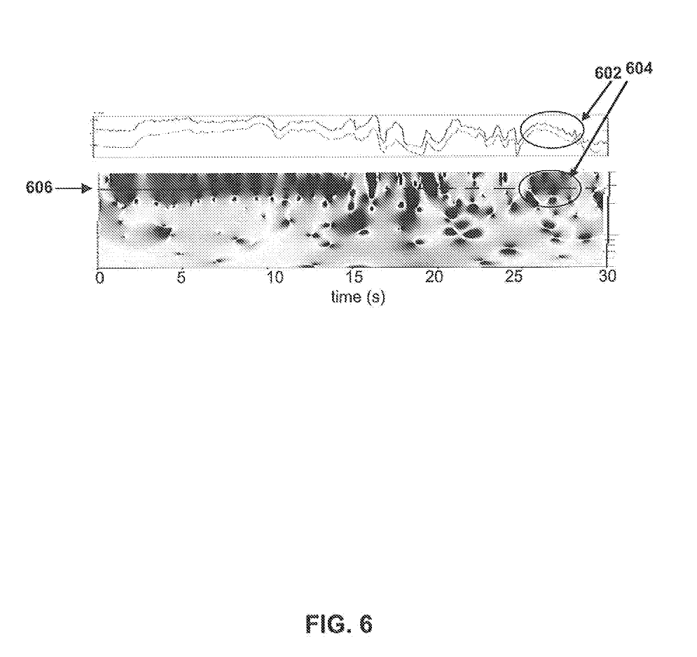

FIG. 6 shows a three-dimensional wavelet ratio surface and corresponding PPG signals 600. The ratio surface includes a stable region (shown in blue) in the vicinity of pulse band 606. As shown in the example of FIG. 6, there is significant artifact present in the wavelet ratio surface from about 15 seconds to about 25 seconds. Another short stable region appears again from about 25 seconds to about 28 seconds, and the artifact reappears after 28 seconds. The short stable region from about 25 seconds to about 28 seconds is evident in both PPG region 602 and wavelet ratio surface region 604. The data modeling processor may be trained to automatically recognize stable regions of the wavelet ratio surface and identify unstable or corrupted regions. The physiological measurement system (e.g., pulse oximetry system) may stop processing surface information when unstable or corrupted regions are detected by the data modeling processor. When a stable region reappears in the wavelet ratio surface, the data modeling processor may signal to start processing surface data. In this way, the data modeling processor may filter the wavelet ratio surface in real-time by identifying windows of valid data. The sizes of the windows may change based, at least in part, on the stable regions identified in the wavelet ratio surface.

The data modeling processor may gate or operate on valid signal segments as the segments are received (e.g., from a streaming data source) or may gate or operate on valid segments in chunks or discrete windows. Regardless of how the data modeling processor operates on incoming data in an embodiment, the data modeling processor may gate signal segments in real-time so that physiological parameter measurements may be outputted continuously using gated data (or previously known-good data). Because scalogram or wavelet ratio surface data may be gated for use at unpredictable times (e.g., whenever a non-corrupted signal segment is detected), all or a part of the gated data may be buffered or stored in memory until new gated data is available. In an embodiment, the buffered data is used in physiological parameter measurements until new gated data becomes available. As described above, however, in other embodiments, extrapolation techniques may be used (e.g., linear or nonlinear regression techniques) in order to extrapolate new data from previously received data. This extrapolated data may additionally or alternatively used in physiological parameter measurements until new gated data becomes available.

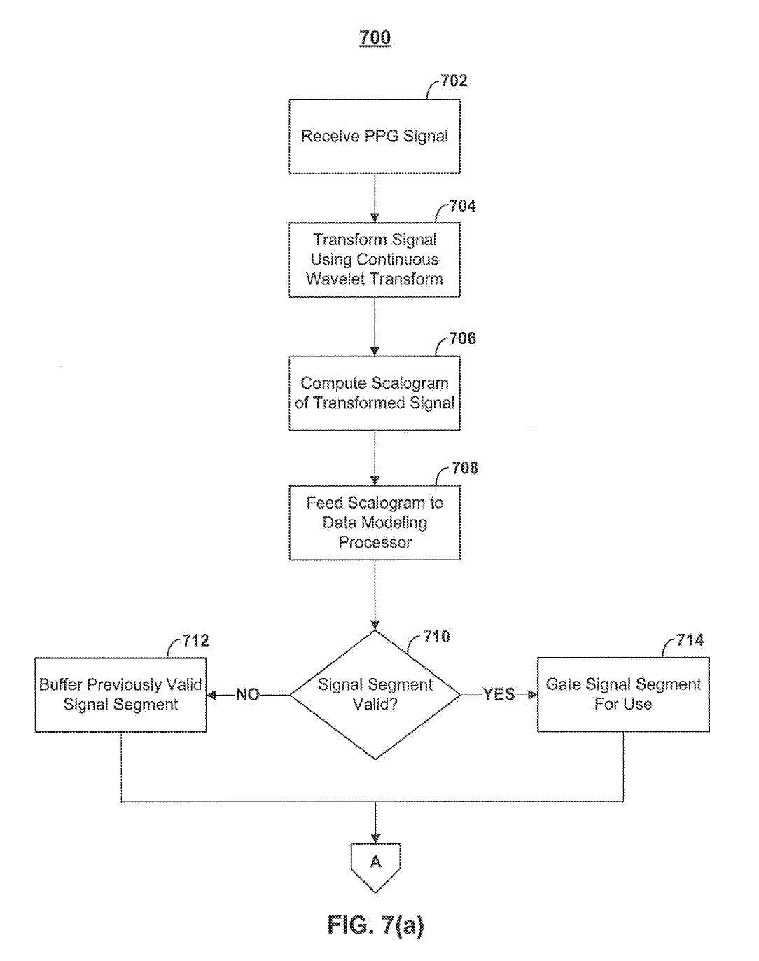

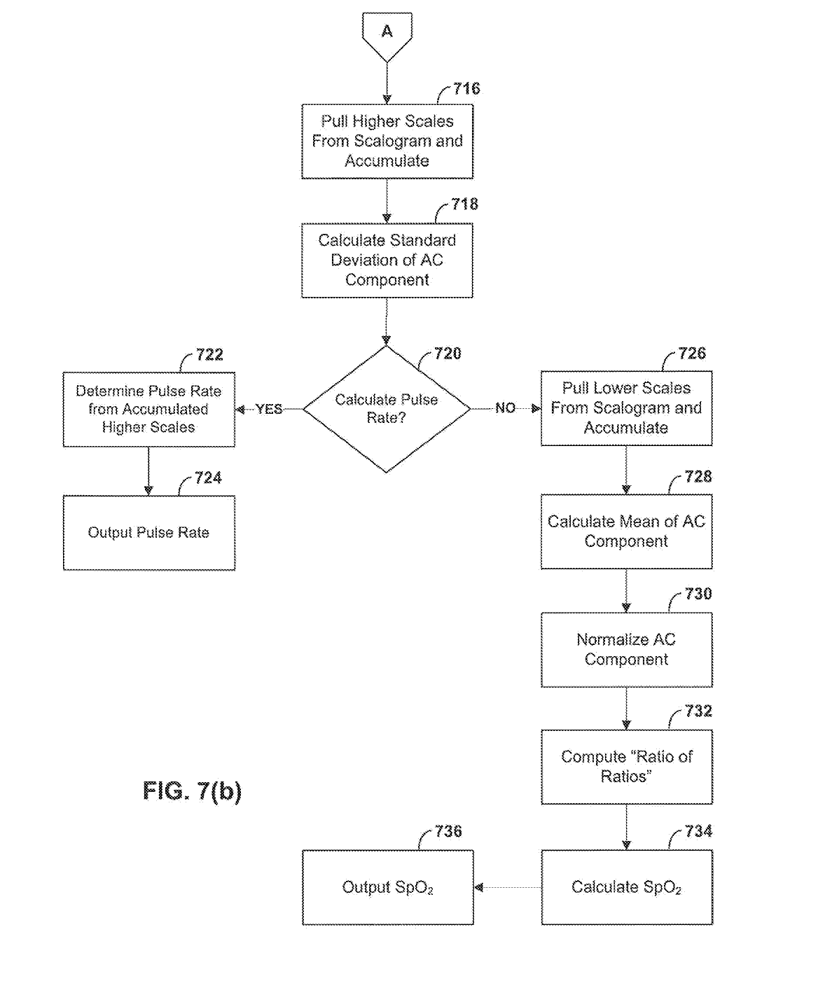

FIGS. 7(a) and 7(b) show illustrative process 700 for determining at least one physiological parameter in accordance with the present disclosure. At step 702, a PPG signal may be received. For example, sensor 12 (FIG. 2) may detect a red signal, an infrared signal, or both a red signal and infrared signal from patient 40 (FIG. 2). At step 704, the received signal may be transformed using, for example, a continuous wavelet transform. In an embodiment, microprocessor 48 (FIG. 2) may perform the transform. At step 706, a scalogram may be computed from the transformed signal. In an embodiment, microprocessor 48 (FIG. 2) may compute the scalogram. At step 708, the scalogram signal may be fed to a data modeling processor. As described above, in some embodiments, the data modeling processor may learn valid characteristics of the scalogram using an artificial neural network. The data modeling processor may then detect areas of increased artifact in the scalogram using any suitable method (e.g., regression analyses, pattern matching, non-linear statistical modeling, or any combination of the foregoing).

At step 710, the data modeling processor may then determine whether the current signal segment of the scalogram contains valid data. As described above, in some embodiments, valid signal segments may include segments not identified as having artifact (or having less than some threshold level of artifact), segments that are not stale (e.g., segments collected within some user-defined freshness threshold), or segments that are both free from artifact and not stale. Tithe current signal segment is valid, the data modeling processor may gate the valid segment for use in determining at least one physiological parameter at step 714. If the current signal segment is not valid, the data modeling processor may buffer a previously valid signal segment at step 712.

As described above, the data modeling processor may operate on discrete windows of data (e.g., a window of M samples by N scales of a scalogram) or may operate on a stream of data as it is received. In addition, in some embodiments, the data modeling processor may operate directly on the PPG signal itself (e.g., before transforming the signal), a wavelet ratio surface, the real part of the wavelet transform, the imaginary part of the wavelet transform, the modulus of the wavelet transform, the energy density of the wavelet transform, any other wavelet representation, or any combination of the foregoing signals. The data modeling processor input may also be a parameterized version of any of the foregoing signals in some embodiments.

Illustrative process 700 continues in FIG. 7(b). At step 716, the higher order scales may be pulled from the scalogram and accumulated until a suitable segment size is collected. The higher order scales pulled from the scalogram may correspond to or include the pulse band (e.g., the scales corresponding to the expected pulse rate). At step 718, the standard deviation of the AC component of the higher order scales may be computed by, for example, microprocessor 48 (FIG. 2). If the pulse rate is to be determined at step 720, then the higher order scales of the accumulated scalogram segments are analyzed and used to determine the pulse rate at step 722. For example, as described above, by employing a suitable resealing of the scalogram segments, the ridges found in wavelet space may be related to the instantaneous characteristic frequency of the signal. In this way, the pulse rate may be obtained directly from the scalogram. Any other suitable technique for determining the pulse rate may also be used at step 722. At step 724, pulse rate measurements may be outputted in real-time. For example, the pulse rate may be outputted on display 20 (FIG. 2). If a pulse rate measurement becomes stale (e.g., relies on buffered data outside some threshold time window), an alarm (e.g., audible or visual alarm) may be automatically triggered.

If, at step 720, pulse rate is not to be determined, at step 726 the lower order scales may be pulled from the scalogram and accumulated until a suitable segment size is collected. At step 728, the mean of the AC component of the lower order scales may be computed by, for example, microprocessor 48 (FIG. 2). At step 730, the AC component signal (of the higher order scales, the lower order scales, or all scales) may be normalized using, for example, the DC component of the accumulated scalogram segments or some baseline signal. In an embodiment, the AC component signal may be divided by the DC component signal or a baseline signal to yield the normalized AC component signal at step 730. At step 732, a “ratio of ratios” may then be computed by dividing, for example, the normalized red AC component signal by the normalized infrared AC component signal, using, for example, equation (4) above.

At step 734, measurements for SpO₂ may then be computed using the ratio computed in step 732. For example, microprocessor 48 (FIG. 2) may determine SpO₂ in accordance with equation (5). The SpO₂ values may then be outputted at step 736. For example, SpO₂ may be outputted on display 20 (FIG. 2). If an SpO₂ measurement becomes stale (e.g., relies on buffered data outside some threshold time window), an alarm (e.g., audible or visual alarm) may be automatically triggered.

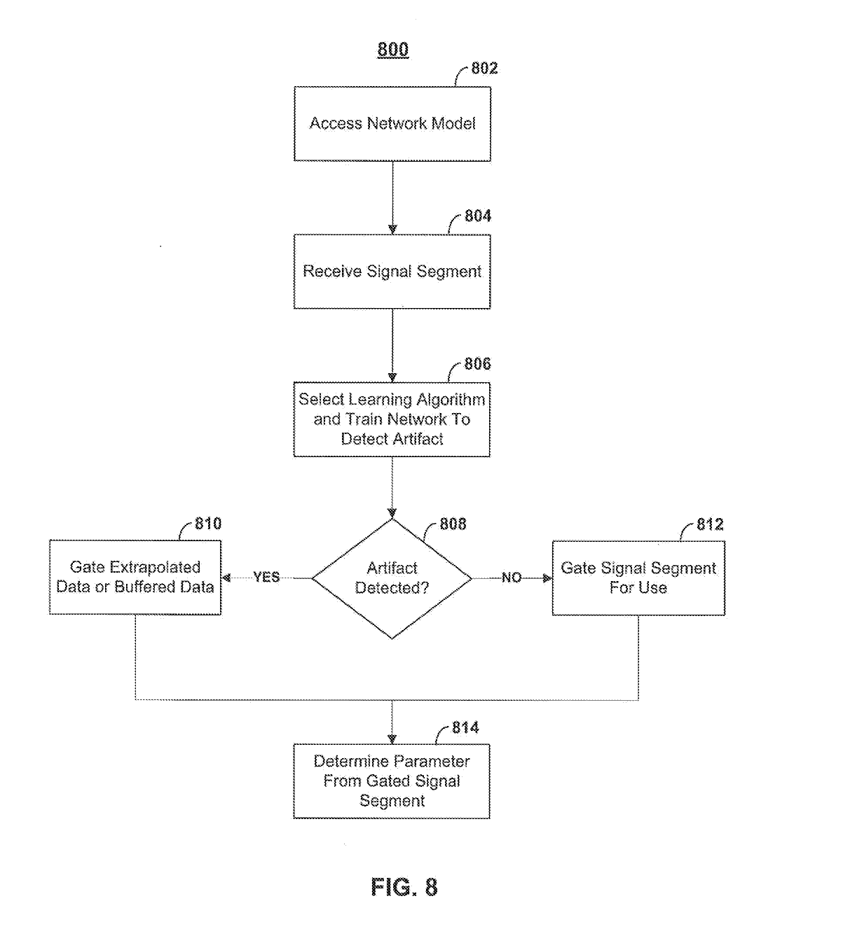

FIG. 8 shows illustrative process 800 for determining a physiological parameter using an artificial neural network in accordance with an embodiment. At step 802, a model is accessed for the neural network. For example, monitor 14 (FIG. 2) may access one or more of a plurality of models stored in RAM 54 and ROM 52 (both of FIG. 2). The choice of model may depend, for example, at least in part on the particular physiological parameter or parameters being determined and the data representation used. As previously described, a data modeling processor implementing the neural network may take as input a PPG signal, a transformed PPG signal (e.g., transformed using a continuous wavelet transform), or any other suitable wavelet representation. In some embodiments, the data modeling processor may operate on a scalogram derived from the transformed signal, a wavelet ratio surface, the real part of the wavelet transform, the imaginary part of the wavelet transform, the modulus of the wavelet transform, the energy density of the wavelet transform, or any combination of the foregoing signals. In general, the model accessed at step 802 may be represented as a composition of functions that are each, in turn, defined by a composition of other functions. In some embodiments, a nonlinear weighted sum is used for the composition. Any other type of composition may also be used.

At step 804, a signal segment or segments may be received. For example, detector 18 (FIG. 2) may receive an optical signal from emitter 16 (FIG. 2). As described above, the received signal segment or segments may then be transformed, in some embodiment, using, for example, a continuous wavelet transform before being passed to the neural network. Signal segments may be received one at a time, or a continuous stream of signal segments may be received. At step 806, a learning technique is selected and the neural network is trained to detect artifacts in the signal segments (or transformed signal segments) using the learning technique. As described above, the learning technique may implement supervised learning, unsupervised learning, reinforcement learning, or any combination of the foregoing learning techniques. Depending on which learning technique or techniques are used, an appropriate cost function may also be defined at step 806. In some embodiments, an arbitrary ad hoc cost function may be used or the posterior probability of the model may be used as an inverse cost function. The cost function may additionally or alternatively be based, at least in part, on the type of artifact to be detected and the physiological parameter to be determined. In general, the model, cost function, and learning technique for the neural network may be selected to maintain robustness of the monitoring system as a whole (e.g., monitor 14 (FIG. 2)).

At step 808, the neural network may determine if the current signal segment or segments contains artifact. This determination may be based, at least in part, on the model selected at step 802 and the training performed in step 806. If artifact is not detected, then the signal segment may be gated for use at step 812. If artifact is detected, however, at step 810 extrapolated data or previously received data may be gated for use. As described above, previous known-good signal segments may be buffered until another artifact-free signal segment is received. Additionally or alternatively, data may be extrapolated from previous data using, for example, a regression analysis and the model accessed in step 802. After a suitable length of gated segments is received, at least one physiological parameter may be determined at step 814. For example, pulse rate or oxygen saturation may be determined in accordance with illustrative process 700 (FIGS. 7(a) and 7(b)). Blood pressure may be determined in accordance with the systems and methods described in U.S. patent application Ser. No. 12/242,238, filed Sep. 30, 2008, entitled “Systems and Methods for Non-Invasive Blood Pressure Monitoring,” which is hereby incorporated by reference herein in its entirety. Respiration rate and respiratory effort may be determined in accordance with the systems and methods described in U.S. patent application Ser. No. 12/245,366, filed Oct. 3, 2008, entitled “Systems and Methods for Determining Effort,” which is hereby incorporated by reference herein in its entirety. Until a suitable length of gated segments is received, previous physiological parameter values may be maintained. In an embodiment, an alarm (e.g., an audible or visual alarm) may be automatically triggered when a physiological parameter value becomes stale (e.g., is based on data received outside some user-defined or system-defined time window).

The neural network implemented by the data modeling processor may include any suitable type of artificial neural network or networks. For example, the neural network or networks may include one or more of a feedforward network, a radial based function (RBF) network, a Kohonen self-organizing network, a recurrent network (e.g., a simple recurrent network, a Hopfield network, an echo state network, or a long short term memory network), a stochastic network, a modular network, an associative network, an instantaneously trained network, a spiking network, a dynamic network, and a cascading network.

The foregoing is merely illustrative of the principles of this disclosure and various modifications can be made by those skilled in the art without departing from the scope and spirit of the disclosure. The above described embodiments are presented for purposes of illustration and not of limitation. The present disclosure also can take many forms other than those explicitly described herein. Accordingly, it is emphasized that the disclosure is not limited to the explicitly disclosed methods, systems, and apparatuses, but is intended to include variations to and modifications thereof which are within the spirit of the following claims.

What is claimed is:

1. A method for determining a physiological parameter, comprising:
receiving, from a sensor, a PPG signal;
using processing circuitry to:
transform the received PPG signal using a continuous wavelet transform,
pass a representation of the transformed signal to a neural network,
detect, with the neural network, a region of artifact in the representation of the transformed signal, and
determine a physiological parameter based at least in part on the representation of the transformed signal and information regarding the region of artifact; and
outputting to an output device the physiological parameter.

2. The method of claim 1 wherein the representation of the transformed signal comprises a scalogram of the transformed signal.

3. The method of claim 1 wherein the representation of the transformed signal comprises a three-dimensional ratio surface of the transformed signal.

4. The method of claim 1 wherein the neural network detects a region of artifact in the representation of the transformed signal by accessing a model for the neural network, the model based, at least in part, on the representation of the transformed signal.

5. The method of claim 1 wherein the neural network detects a region of artifact in the representation of the transformed signal by selecting a learning algorithm for the neural network, the learning algorithm implementing at least one of supervised learning, unsupervised learning, and reinforcement learning.

6. The method of claim 5 further comprising training the neural network to detect artifact in the representation of the transformed signal using the learning algorithm.

7. The method of claim 1 further comprising using the processing circuitry to modify the representation of the transformed signal by removing the detected region of artifact from the representation of the transformed signal.

8. The method of claim 1 further comprising using the processing circuitry to modify the representation of the transformed signal by replacing the detected region of artifact in the representation of the transformed signal with extrapolated data.

9. The method of claim 1 further comprising using the processing circuitry to modify the representation of the transformed signal by replacing the detected region of artifact with previously received buffered data.

10. The method claim 1 wherein the processing circuitry determines a pulse rate from the representation of the transformed signal.

11. A system for determining a physiological parameter, comprising:
a sensor configured to receive a PPG signal; and
processing circuitry configured to:
transform the received PPG signal using a continuous wavelet transform;
pass a representation of the transformed signal to a neural network;
detect, with the neural network, a region of artifact in the representation of the transformed signal; and
determine a physiological parameter based at least in part on the representation of the transformed signal and information regarding the region of artifact.

12. The system of claim 11 further comprising an output device to output the physiological parameter.

13. The system of claim 11 wherein the representation of the transformed signal comprises a scalogram of the transformed signal.

14. The system of claim 11 wherein the representation of the transformed signal comprises a three-dimensional ratio surface of the transformed signal.

15. The system of claim 11 wherein the neural network is configured to detect a region of artifact in the representation of the transformed signal by accessing a model for the neural network, the model based, at least in part, on the representation of the transformed signal.

16. The system of claim 11 wherein the neural network is configured to detect a region of artifact in the representation of the transformed signal by selecting a learning algorithm for the neural network, the learning algorithm implementing at least one of supervised learning, unsupervised learning, and reinforcement learning.

17. The system of claim 16 wherein the processing circuitry is configured to train the neural network to detect artifact in the representation of the transformed signal using the learning algorithm.

18. The system of claim 11 wherein the processing circuitry is configured to modify the representation of the transformed signal by removing the detected region of artifact from the representation of the transformed signal.

19. The system of claim 11 wherein the processing circuitry is configured to modify the representation of the transformed signal by replacing the detected region of artifact in the representation of the transformed signal with extrapolated data.

20. The system of claim 11 wherein the processing circuitry is configured to modify the representation of the transformed signal by replacing the detected region of artifact with previously received buffered data.
